(19) United States
(12) Patent Application Publication
(10) Pub. No.: US 2024/0003844 A1
(43) Pub. Date: Jan. 4, 2024

(54) BIOSENSOR, DETECTION METHOD, AND DETECTION DEVICE

(71) Applicants: Yasuo SUZUKI, Kyoto-shi (JP); Murata Manufacturing Co., Ltd., Nagaokakyo-shi (JP); OSAKA UNIVERSITY, Osaka (JP)

(72) Inventors: Yasuo SUZUKI, Kyoto-shi (JP); Shota USHIBA, Nagaokakyo-shi (JP); Naruto MIYAKAWA, Nagaokakyo-shi (JP); Takao ONO, Suita-shi (JP); Kazuhiko MATSUMOTO, Suita-shi (JP)

(21) Appl. No.: 18/368,179

(22) Filed: Sep. 14, 2023

Related U.S. Application Data
(63) Continuation of application No. PCT/JP2022/004686, filed on Feb. 7, 2022.

(30) Foreign Application Priority Data
Mar. 15, 2021 (JP) 2021-041876

(51) Int. Cl. G01N 27/414

(52) U.S. Cl. CPC G01N 27/4145; G01N 27/4146

(57) ABSTRACT

A biosensor is a field-effect transistor-based biosensor including an insulating substrate, and a measurement sensor and a reference sensor on the insulating substrate. A probe molecule is in the measurement sensor. The probe molecule has a first basic moiety in the measurement sensor, and a recognition moiety with a first end bound to the first basic moiety and a second end defining a distal end of the probe molecule. A second basic moiety is in the reference sensor and has a same structure as that of the first basic moiety of the probe molecule in the measurement sensor. The recognition moiety of the probe molecule in the measurement sensor is absent at the distal end of the second basic moiety.

### CROSS REFERENCE TO RELATED APPLICATIONS

This application claims the benefit of priority to Japanese Patent Application No. 2021-041876 filed on Mar. 15, 2021 and is a Continuation Application of PCT Application No. PCT/JP2022/004686 filed on Feb. 7, 2022. The entire contents of each application are hereby incorporated herein by reference.

### BACKGROUND OF THE INVENTION

### 1. Field of the Invention

The present invention relates to a biosensor, a detection method, and a detection device.

### 2. Description of the Related Art

A highly sensitive detection method enabling quick detection of a target substance for detection (also referred to as target molecule) such as a cell, a microorganism, a virus, a protein, or a nucleic acid (DNA or RNA) is desired.

For example, it is known that viruses such as influenza virus each infect hosts by recognizing and binding to a specific glycan on the surfaces of host cells.

JP 2017-121205 A discloses a solid phase to which an α2,3-linked sialic acid-including glycan and/or an α2,6-linked sialic acid-including glycan is fixed. JP 2017-121205 A also discloses a virus classification method including a step of contacting a virus with a solid phase to which an α2,3-linked sialic acid-including glycan and/or an α2,6-linked sialic acid-including glycan is fixed, a step of detecting the virus bound to the solid phase, and a step of classifying the virus based on the binding activity of the virus to the α2,3-linked sialic acid-including glycan or the α2,6-linked sialic acid-including glycan. According to JP 2017-121205 A, because the binding form of a saccharide and the sialic acid to which the virus can bind varies depending on the type of viruses, the virus can be classified by examining the type of sialic acid-including glycans to which the virus binds.

According to JP 2017-121205 A, preferably, an asialoglycan is further fixed to the solid phase disclosed in JP 2017-121205 A. In JP 2017-121205 A, because the virus does not bind to the asialoglycan, the region of the solid phase to which the asialoglycan is fixed can be used as a negative control of the reaction.

Recently, field-effect transistor (FET)-based biosensors are proposed as a biosensor using a probe molecule which specifically interacts with a target molecule which is a target substance for detection.

T. Ono et al. Jpn. J. Appl. Phys. 56, 030302 (2017) discloses a graphene FET-based biosensor including graphene functionalized by glycan modification. T. Ono et al. Jpn. J. Appl. Phys. 56, 030302 (2017) demonstrates the principle of the sensor using lectin having glycan-binding properties similar to those of influenza virus, reporting that the virus can be highly selectively detected with high sensitivity by functionalizing graphene with an α2,6-linked glycan or an α2,3-linked glycan.

JP 6782218 B discloses a detection device including a sensor element, and a probe molecule which is fixed to the sensor element and associates with a receptor exposed on the surface of a detection target, wherein the sensor element detects cleavage of the receptor by a specific protease, the receptor being associated with and trapped by the probe molecule. According to JP 6782218 B, influenza virus can be detected using an α2,6-linked glycan or an α2,3-linked glycan as the probe molecule.

While such FET-based biosensors disclosed in T. Ono et al. Jpn. J. Appl. Phys. 56, 030302 (2017) and JP 6782218 B are promising, accuracy and sensitivity thereof are likely to vary in sensing using a biological fluid including the target molecule and the like. This is because biological fluids including the target molecule vary among individuals, and for example, the concentration of the electrolyte, the concentration of molecules other than the target molecule, such as proteins and enzymes, and the temperature vary. This leads to a demand for a method of controlling these variations.

JP 6190355 B discloses a device which senses the presence of a specific target molecule or a biomarker in a sample by detecting a change in electrical properties, the device including a measurement sensor including a coating of an aptamer capable of conjugating with the biomarker, and a sensor capped so as not to conjugate with the biomarker in order to cause the sensor to act as an internal reference. According to JP 6190355 B, the capping is effected by saturation of a sensor structure with the target molecule or the biomarker in advance; the sensor structure includes an oligonucleotide aptamer, and the capping is effected by binding the sensor structure to its complementary oligonucleotide; or the capping is effected by using a mutant of the sensor structure having a change in sequence such that the mutant no longer recognizes the biomarker. In the detection device disclosed in JP 6190355 B, because the two sensors forming a pair are disposed in the same molecule environment, that is, in the same measurement space, these sensors are equally influenced by the environment. For this reason, a variation attributed to the electrolyte in a fluid sample or the like is reduced by comparison between the signals from the two sensors.

### SUMMARY OF INVENTION

According to JP 6190355 B, the detection device disclosed therein can reduce a variation attributed to noises other than the target molecule as the target substance for detection, by using a reference sensor serving as the internal reference. However, because the technique disclosed in JP 6190355 B is applicable to the case where the aptamer is used as the probe molecule, the technique cannot be used in the case where a glycan is used as the probe molecule, for example. Moreover, in the detection device disclosed in JP 6190355 B, the probe molecule is capped by saturation thereof with the target molecule or the biomarker in advance. However, because the reaction between the probe molecule and the target molecule is a reversible reaction, the capping may be uncapped even if the probe molecule is saturated in advance.

Preferred embodiments of the present invention provide field-effect transistor-based biosensors each enabling a reduction in variations attributed to noises and having high measurement precision and reliability. Preferred embodiments of the present invention also provide detection methods and detection devices each including a biosensor according to a preferred embodiment of the present invention.

A biosensor according to a preferred embodiment of the present invention is a field-effect transistor-based biosensor including an insulating substrate, and a measurement sensor and a reference sensor on the insulating substrate, wherein the measurement sensor includes a first semiconductor layer, a first source electrode, and a first drain electrode, the first source electrode and the first drain electrode being electrically connected to the first semiconductor layer, the reference sensor includes a second semiconductor layer, a second source electrode, and a second drain electrode, the second source electrode and the second drain electrode being electrically connected to the second semiconductor layer, a probe molecule is in the measurement sensor, the probe molecule has a first basic moiety in the measurement sensor, and a recognition moiety with a first end bound to the first basic moiety and a second end defining a distal end of the probe molecule, a second basic moiety is in the reference sensor, the second basic moiety having a same structure as that of the first basic moiety of the probe molecule in the measurement sensor, and the recognition moiety of the probe molecule in the measurement sensor is absent at the distal end of the second basic moiety.

A detection method according to a preferred embodiment of the present invention is a method of detecting a target substance for detection using a biosensor according to a preferred embodiment of the present invention, the method including feeding a sample including a target substance for detection to the measurement sensor and the reference sensor, measuring a value of a first current flowing between the first source electrode and the first drain electrode in the measurement sensor by applying a voltage between the first source electrode and the first drain electrode, measuring a value of a second current flowing between the second source electrode and the second drain electrode in the reference sensor by applying a voltage between the second source electrode and the second drain electrode, and correcting a property of the measurement sensor by comparing a property of the measurement sensor obtained from the value of the first current to a property of the reference sensor obtained from the value of the second current or by comparing a temporal change in the property of the measurement sensor to a temporal change in the property of the reference sensor.

A detection device according to a preferred embodiment of the present invention is a device to detect a target substance for detection using a biosensor according to a preferred embodiment of the present invention, the device including a first current calculator to measure a value of the first current flowing between the first source electrode and the first drain electrode in the measurement sensor by applying a voltage between the first source electrode and the first drain electrode, a second current calculator to measure a value of the second current flowing between the second source electrode and the second drain electrode in the reference sensor by applying a voltage between the second source electrode and the second drain electrode, and a corrector to correct a property of the measurement sensor by comparing a property of the measurement sensor obtained from the value of the first current to a property of the reference sensor obtained from the value of the second current or by comparing a temporal change in the property of the measurement sensor to a temporal change in the property of the reference sensor.

Preferred embodiments of the present invention provide field-effect transistor-based biosensors each enabling a reduction in variations attributed to noises and having high measurement precision and reliability. Preferred embodiments of the present invention also provide detection methods and detection devices each including a biosensor according to a preferred embodiment of the present invention.

The above and other elements, features, steps, characteristics and advantages of the present invention will become more apparent from the following detailed description of the preferred embodiments with reference to the attached drawings.

### BRIEF DESCRIPTION OF DRAWINGS

FIG. 1 is a schematic view showing one example of a biosensor according to a preferred embodiment of the present invention.

FIG. 2 is a schematic view showing the structure of Influenza A virus.

FIG. 3 is a diagram showing one example of the structure of an α2,3-linked sialic acid-including glycan binding peptide.

FIG. 4 is a diagram showing one example of the structure of an α2,6-linked sialic acid-including glycan binding peptide.

FIG. 5 is a diagram showing one example of the structure of an asialoglycan-binding peptide.

FIG. 6 is a schematic view showing a basic structure of an antibody.

FIG. 7 is a schematic view showing the Fab portion and the Fc portion of the antibody.

FIG. 8 is a schematic view showing one example of a step of providing an insulating substrate.

FIG. 9 is a schematic view showing one example of a step of forming source electrodes, drain electrodes, and semiconductor layers.

FIG. 10 is a schematic view showing one example of a step of separating between a measurement sensor region and a reference sensor region.

FIG. 11 is a schematic view showing one example of a step of disposing a probe molecule and a second basic moiety.

FIG. 12 is a schematic view showing another example of a biosensor according to a preferred embodiment of the present invention.

FIG. 13 is a schematic view showing further another example of a biosensor according to a preferred embodiment of the present invention.

FIG. 14 is a schematic view schematically showing one example of a biosensor according to a preferred embodiment of the present invention when used.

FIG. 15 is a graph showing the relation between the gate voltage VG and the source-drain current I_{DS}.

FIG. 16 is a graph showing one example of the temporal change in sensor property.

FIG. 17 is a graph showing one example of the outputs of the measurement sensor and the reference sensor by the detection method using a biosensor according to a preferred embodiment of the present invention.

FIG. 18 is a graph showing one example of the outputs of the measurement sensor and the reference sensor by the detection method using a conventional biosensor.

FIG. 19 is a schematic view showing one example of a biosensor according to a first preferred embodiment of the present invention.

FIG. 20 is a schematic view showing one example of a biosensor according to a second preferred embodiment of the present invention.

FIG. 21 is a diagram showing one example of the structure of the linker molecule including a pyrenyl group.

FIG. 22 is a schematic view showing one example of a biosensor according to a third preferred embodiment of the present invention.

FIG. 23 is a schematic view showing one example of a biosensor according to a fourth preferred embodiment of the present invention.

FIG. 24 is a schematic view showing another example of a biosensor according to the fourth preferred embodiment of the present invention.

FIG. 25 is a schematic view showing one example of a biosensor according to a fifth preferred embodiment of the present invention.

### DETAILED DESCRIPTION OF THE PREFERRED EMBODIMENTS

Hereinafter, biosensors, detection methods, and detection devices according to preferred embodiments of the present invention will be described.

However, the configurations described below should not be construed as limitations on preferred embodiments of the present invention, and can be appropriately modified and applied in the range not changing the gist of the present invention. The present invention also covers combinations of two or more of preferred individual configurations according to the present invention described below.

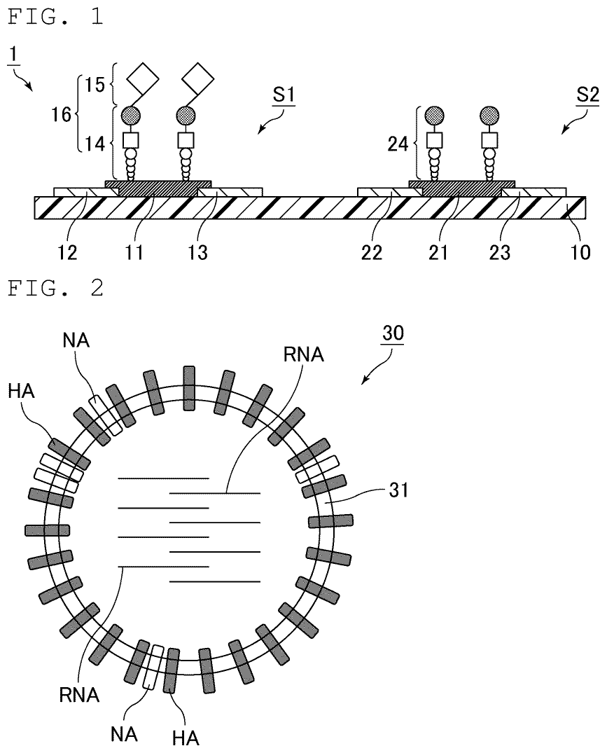

FIG. 1 is a schematic view showing one example of a biosensor according to a preferred embodiment of the present invention.

FIG. 1 shows portions whose sizes, thicknesses, and the like are appropriately changed for clarity and simplicity of the illustrations. The same is applied to other drawings.

A biosensor 1 shown in FIG. 1 is a field-effect transistor (FET)-based biosensor including a measurement sensor 51 and a reference sensor S2 on an insulating substrate 10.

The measurement sensor 51 includes a first semiconductor layer 11, a first source electrode 12, and a first drain electrode 13, the first source electrode 12 and the first drain electrode 13 being electrically connected to the first semiconductor layer 11. In the example shown in FIG. 1, the first source electrode 12 and the first drain electrode 13 are spaced apart from each other on the insulating substrate 10, and the insulating substrate 10 is exposed between the first source electrode 12 and the first drain electrode 13. The first semiconductor layer 11 is disposed on the insulating substrate 10 to cover one end of the first source electrode 12, the exposed portion of the insulating substrate 10, and one end of the first drain electrode 13. The first semiconductor layer 11 between the first source electrode 12 and the first drain electrode 13 defines a channel of the measurement sensor S1.

The measurement sensor S1 includes a probe molecule 16 having a first basic moiety 14 and a recognition moiety 15. The first basic moiety 14 is disposed in the measurement sensor S1. One end of the recognition moiety 15 is bound to the first basic moiety 14, and the other end of the recognition moiety 15 defines the distal end of the probe molecule 16. In the example shown in FIG. 1, a glycan is disposed as the probe molecule 16. The probe molecule 16 is not limited to the glycan, and may be an antibody or an enzyme, for example.

The reference sensor S2 includes a second semiconductor layer 21, a second source electrode 22, and a second drain electrode 23, the second source electrode 22 and the second drain electrode 23 being electrically connected to the second semiconductor layer 21. In the example shown in FIG. 1, the second source electrode 22 and the second drain electrode 23 are spaced apart from each other on the insulating substrate 10, and the insulating substrate 10 is exposed between the second source electrode 22 and the second drain electrode 23. The second semiconductor layer 21 is disposed on the insulating substrate 10 to cover one end of the second source electrode 22, the exposed portion of the insulating substrate 10, and one end of the second drain electrode 23. The second semiconductor layer 21 between the second source electrode 22 and the second drain electrode 23 defines a channel of the reference sensor S2.

A second basic moiety 24 is disposed in the reference sensor S2, the second basic moiety 24 having the same structure as that of the first basic moiety 14 of the probe molecule 16 disposed in the measurement sensor S1. The recognition moiety 15 of the probe molecule 16 disposed in the measurement sensor S1 is absent at the distal end of the second basic moiety 24.

The FET-based biosensor is provided with a mechanism mimicking the living organisms in the channel portion, and detects the reaction occurring there as FET electrical properties. For example, a glycan selectively binding to a virus such as influenza virus is disposed as the probe molecule 16 in the measurement sensor S1. The probe molecule 16 is preferably immobilized to the measurement sensor S1. As long as the solid-phased probe molecule 16 remains on the surface of the solid material, it may be fixed or tightly adhered to the surface of the solid material, or may have a certain degree of freedom to be movable.

Although an example using influenza virus will be described below, other viruses such as reovirus, adenovirus, rotavirus, and novel coronavirus (SARS-CoV2) can be detected by selecting an appropriate glycan.

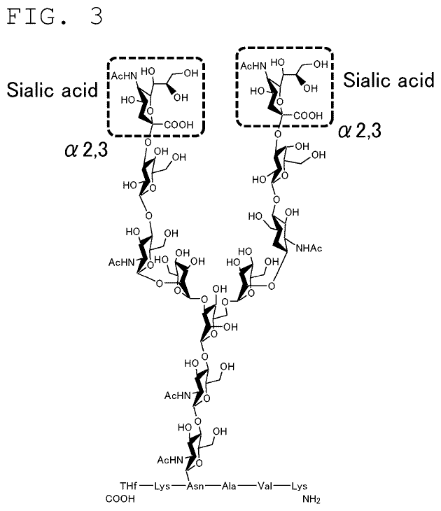

FIG. 2 is a schematic view showing the structure of Influenza A virus.

The influenza virus 30 is a particle having a diameter of about 80 nm or more and about 120 nm or less, for example, and surrounded with a lipid bilayer (surface membrane) called envelope 31, and has single-stranded RNA (ribonucleic acid) genome inside the envelope 31. Two spike proteins (proteins sticked into the membrane) having different lengths are exposed on the surface of the envelope 31. These are called hemagglutinin (hereinafter, abbreviated to HA) and neuraminidase (hereinafter, abbreviated to NA).

Influenza virus has types A, B, C, and D. Types A and B have HA and NA, and are not much different in terms of structure. Types C and D have one spike protein called hemagglutinin-esterase-fusion (HEF) playing roles of both HA and NA, instead of HA and NA.

Type C mainly infects children aged 4 or younger. Type C barely infects animals other than humans. Type B also barely infects animals other than humans. In Type A, there are some subtypes which infect animals in addition to humans.

Among subtypes of Influenza A virus, some infects birds, and some infects humans and domestic and wild animals (such as tigers, dogs, and common raccoons). This is attributed to a difference between birds and humans in molecular structure of the glycan exposed on the surface of the upper respiratory cell to which influenza virus first adsorbs. When influenza virus adsorbs to the cell, HA exposed on the surface of the virus recognizes and binds to the glycan exposed on the surface of the cell.

It is known that the glycan exposed on the bird respiratory tract mainly has an α2,3 structure, and the glycan exposed from the human respiratory tract mainly has an α2,6 structure. The difference between these comes from a different carbon position at which the sialic acid located at the distal end of the glycan and galactose next to the sialic acid are bound to each other. Therefore, it is known that usually, avian Influenza A virus binds to a glycan terminated with sialic acid bound to galactose via an α2,3 bond (hereinafter, also referred to as α2,3-linked glycan), and human Influenza A virus binds to a glycan terminated with sialic acid bound to galactose via an α2,6 bond (hereinafter, also referred to as α2,6-linked glycan).

It is also known that human Influenza B virus binds to a glycan terminated with sialic acid bound to galactose via an α2,6 or α2,3 bond.

Thus, it is known that in many viruses including influenza virus, their surface HA recognizes the sialic acid at the distal end of a specific glycan, and binds to the specific glycan.

In this specification, “sialic acid” means a general name for substances in which an amino group and/or a hydroxy group of neuraminic acid, which is a nonose, is substituted. Examples of sialic acid include N-acetylneuraminic acid (Neu5Ac) acetylated at position 5, and N-glycolylneuraminic acid (Neu5Gc) modified with glycolic acid at position 5.

As described above, in the biosensor 1 shown in FIG. 1, the probe molecule 16 is disposed in the measurement sensor S1, and has the recognition moiety 15 which selectively binds to the target molecule at its distal end. This can detect a specific target molecule.

On the other hand, the second basic moiety 24 is disposed in the reference sensor S2. The second basic moiety 24 is preferably immobilized to the reference sensor S2. Because the recognition moiety 15 is absent at the distal end of the second basic moiety 24, a signal derived from the target molecule is not detected in the reference sensor S2. In contrast, the reference sensor S2 can detect noises other than the target molecule. For this reason, the reference sensor S2 can be used for correction of noises to the measurement sensor S1.

For example, in the case where an α2,6-linked glycan that binds to human Influenza A virus is disposed as the probe molecule 16 in the measurement sensor S1, an α2,3-linked glycan that does not bind to human Influenza A virus may be disposed in the reference sensor S2. However, in the case where human Influenza B virus or the like which reacts with the α2,3-linked glycan is included as impurities, the α2,3-linked glycan does not act as the reference sensor S2 because these impurities react with the α2,3-linked glycan.

In contrast, in the case where an asialoglycan not having sialic acid as the recognition moiety 15 at the distal end is disposed as the second basic moiety 24 in the reference sensor S2, the risk of cross-reacting with impurities can be reduced because the asialoglycan has no binding ability to the virus. Furthermore, because the second basic moiety 24 and the first basic moiety 14 of the probe molecule 16 have the same structure, these basic moieties have a similar response to noises other than the target molecule. Such a configuration allows the second basic moiety 24 to sufficiently function as the reference sensor S2.

Thus, by comparing the output of the measurement sensor S1 to that of the reference sensor S2, a variation attributed to noises can be reduced, thus enhancing the measurement precision and reliability.

As described above, the probe molecule 16 is a glycan, for example. The virus as the target molecule can be detected by disposing the glycan as the probe molecule 16 in the measurement sensor S1. In the case where the probe molecule 16 is a glycan, the first basic moiety 14 and the second basic moiety 24 both are glycans having the same structure. As one example where the first basic moiety 14 and the second basic moiety 24 both are glycans, the first basic moiety 14 and the second basic moiety 24 both are an asialoglycan, and a probe molecule 16 is a sialoglycan.

In this specification, the term “asialoglycan” means a glycan in which sialic acid is not added to the non-reducing terminal of the glycan structure. The glycans in the living organisms are present mainly in the form of glycoprotein, glycolipid, or oligosaccharide. In this case, examples thereof include an asialoglycan in which galactose is present in the non-reducing terminal. In contrast, the term “sialoglycan” means a glycan in which sialic acid is added to the non-reducing terminal of the glycan structure.

The sialoglycan is, for example, an α2,3-linked sialic acid-including glycan and/or an α2,6-linked sialic acid-including glycan.

The α2,3-linked sialic acid-including glycan may be an α2,3-linked sialic acid-including glycan binding peptide, the α2,6-linked sialic acid-including glycan may be an α2,6-linked sialic acid-including glycan binding peptide, and the asialoglycan may be an asialoglycan-binding peptide. In this case, using peptides, the first basic moiety 14 and the second basic moiety 24 can be fixed to the measurement sensor S1 and the reference sensor S2, respectively. These peptides can have any length.

FIG. 3 is a diagram showing one example of the structure of the α2,3-linked sialic acid-including glycan binding peptide.

FIG. 3 shows α2,3-sialylglycopeptide (α2,3-SGP). As shown in FIG. 3, sialic acid α2,3-linked to galactose is present at the distal end of the glycan.

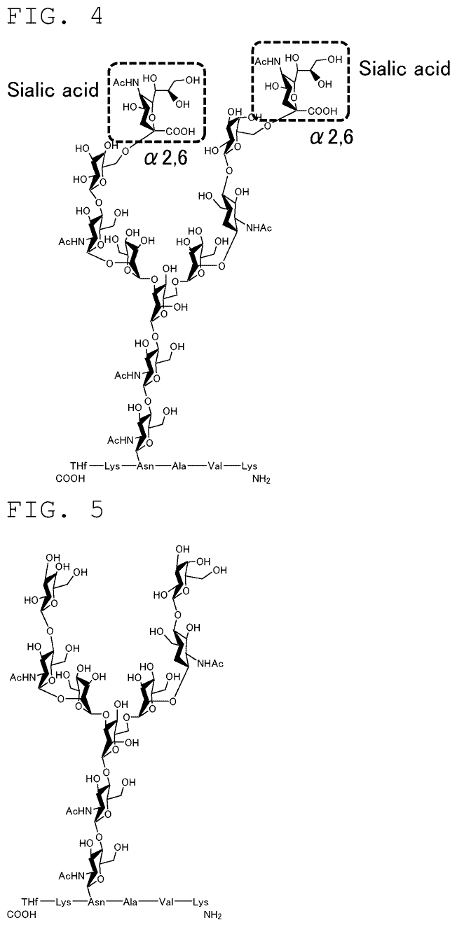

FIG. 4 is a diagram showing one example of the structure of the α2,6-linked sialic acid-including glycan binding peptide.

FIG. 4 shows α2,6-sialylglycopeptide (α2,6-SGP). As shown in FIG. 4, sialic acid α2,6-linked to galactose is present at the distal end of the glycan.

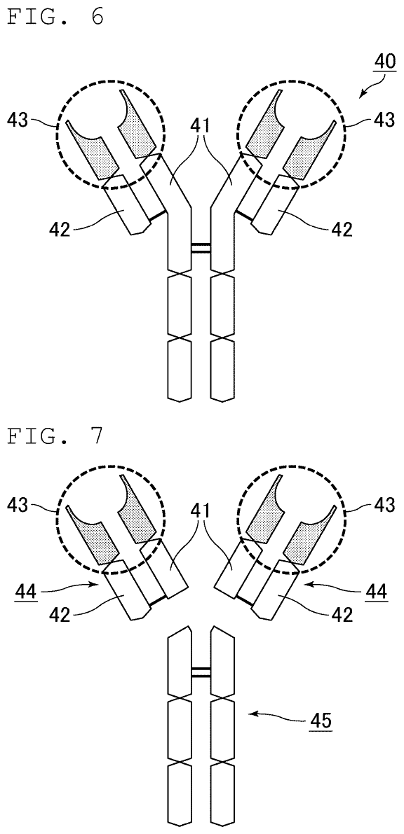

FIG. 5 is a diagram showing one example of the structure of the asialoglycan-binding peptide.

FIG. 5 shows asialoglycopeptide. Unlike FIGS. 3 and 4, sialic acid is absent at the distal end of the glycan.

As described above, the probe molecule 16 may be an antibody or an enzyme.

FIG. 6 is a schematic view showing the basic structure of an antibody.

An antibody 40 has a Y shape formed by four linear polypeptides, i.e., two heavy chains (H chains) 41 and two light chains (L chains) 42 bound via a disulfide bond and a non-covalent bond. An antigen-binding site 43 specifically binding to an antigen is present at the distal end of a V shaped portion located in the upper half of the Y-shaped portion.

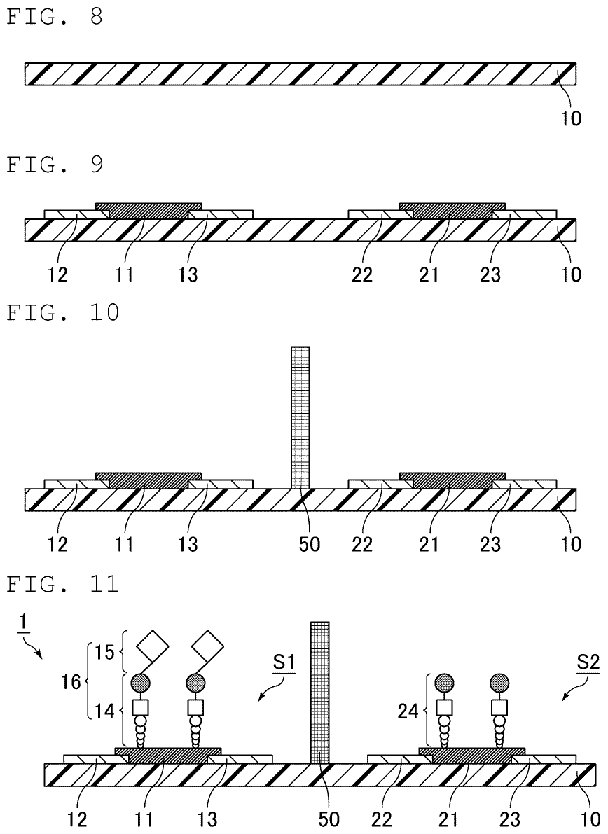

FIG. 7 is a schematic view showing a Fab portion and an Fc portion of the antibody.

As shown in FIG. 7, it is known that the antibody 40 is decomposed into two Fab portions 44 and one Fc portion 45 by papain as a protease. Although not shown, it is known that the antibody 40 is decomposed into one F(ab′)2 portion and many Fc portions by pepsin as another protease. The Fab portion and the F(ab′)2 portion, which have the antigen-binding site, have specificity to an antigen, and can be used in the antigen-antibody reaction.

In the case where the probe molecule 16 is an antibody, the first basic moiety 14 and the second basic moiety 24 are both Fc portions having the same structure.

Examples of the insulating substrate 10 include a thermally oxidized silicon substrate having an oxidized silicon (SiO₂) layer formed by oxidizing the surface of a silicon (Si) substrate, and a boron nitride (BN) substrate. The insulating substrate 10 can be made of any material without limitation, and an inorganic compound such as silicon oxide, silicon nitride, aluminum oxide, titanium oxide, or calcium fluoride, or an organic compound such as an acrylic resin, polyimide, or a fluorinated resin is used, for example. The insulating substrate 10 can be of any shape, which may be a shape of a flat plate or a curved plate. The insulating substrate 10 may have flexibility.

The first semiconductor layer 11 preferably includes graphene or carbon nanotubes. Use of an FET-based transistor with a channel made of graphene or carbon nanotubes can provide a biosensor having enhanced sensitivity.

Graphene is a two-dimensional material of carbon atoms bonded into a hexagonal mesh. Graphene has a very large specific surface area (surface area per volume), and has very high electrical mobility.

Carbon nanotubes are long tubular carbon compounds. As the carbon nanotubes, single-walled carbon nanotubes (SW-CNTs) including a single carbon layer with a mesh structure similar to that of graphene may be used, or multi-walled carbon nanotubes (MW-CNTs) each including many carbon layers stacked may be used. Both of the carbon nanotubes have high conductivity.

The first semiconductor layer 11 may include a two-dimensional material other than graphene or the carbon nanotubes. Specific examples of two-dimensional materials other than graphene include molybdenum disulfide and boron nitride.

The number of layers of the first semiconductor layer 11 is not limited to one, and the first semiconductor layer 11 may be two layers or three or more layers. The first semiconductor layer 11 includes preferably ten or less layers, more preferably five or less layers. The number of layers need not be the same in the entire first semiconductor layer 11, and for example, a single layer portion and two or more layer portions may coexist in first semiconductor layer 11. The number of layers of the first semiconductor layer 11 can be measured by Raman spectroscopy or observation of a cross-section with a transmission electron microscope (TEM).

The second semiconductor layer 21 preferably includes graphene or carbon nanotubes. The second semiconductor layer 21 may include a two-dimensional material other than graphene, instead of graphene or carbon nanotubes. The material included in the second semiconductor layer 21 and the material included in the first semiconductor layer 11 may be the same or different.

The number of layers of the second semiconductor layer 21 is not limited to one, and the second semiconductor layer 21 may be two layers or three or more layers. The number of the layers of the second semiconductor layer 21 is preferably ten or less layers, more preferably five or less layers. The number of layers need not be the same in the entire second semiconductor layer 21, and for example, a single layer portion and two or more layer portions may coexist in the second semiconductor layer 21. The number of the layers of the second semiconductor layer 21 and that of layers of the first semiconductor layer 11 may be the same or different.

The first source electrode 12 and the first drain electrode 13 are, for example, electrodes having a multi-layered structure in which a titanium (Ti) layer and a gold (Au) layer are stacked. As the electrode material, besides titanium and gold, for example, a monolayer of a metal such as gold, platinum, titanium, or palladium may be used, or two or more metals may be used in combination in the form of a multi-layered structure.

The second source electrode 22 and the second drain electrode 23 are, for example, electrodes having a multi-layered structure in which a titanium (Ti) layer and a gold (Au) layer are stacked. As the electrode material, besides titanium and gold, for example, a monolayer of a metal such as gold, platinum, titanium, or palladium may be used, or two or more metals may be used in combination in the form of a multi-layered structure. The material for the second source electrode 22 and the second drain electrode 23 and those for the first source electrode 12 and the first drain electrode 13 may be the same or different.

Hereinafter, one example of the method of producing the biosensor 1 shown in FIG. 1 will be described.

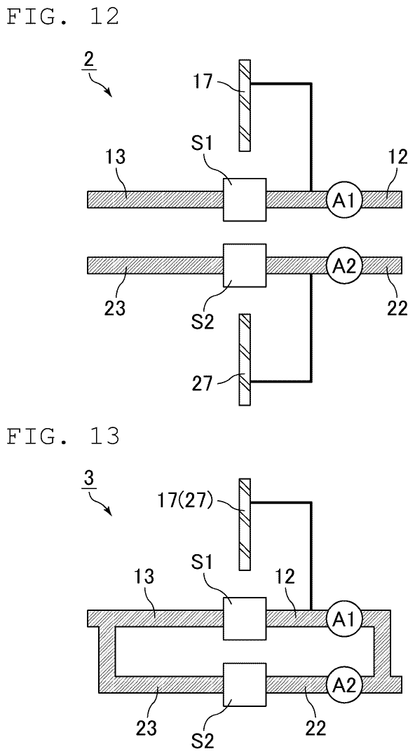

FIG. 8 is a schematic view showing one example of the step of providing an insulating substrate.

As shown in FIG. 8, an insulating substrate 10 is provided. As the insulating substrate 10, a thermally oxidized silicon substrate having an oxidized silicon layer formed by oxidizing the surface of a silicon substrate is used, for example.

FIG. 9 is a schematic view showing one example of a step of forming source electrodes, drain electrodes, and semiconductor layers.

Initially, an electrode pattern is formed on the surface of the insulating substrate 10 by a standard photolithographic process.

For example, a Ti layer and an Au layer are formed on the insulating substrate 10 using a method such as vacuum evaporation, electron beam (EB) deposition, or sputtering. Patterning is then performed by photolithography and etching to form the first source electrode 12, the first drain electrode 13, the second source electrode 22, and the second drain electrode 23.

Subsequently, semiconductor layers are formed on the surface of the insulating substrate 10 on which an electrode pattern is formed.

For example, a two-dimensional material such as graphene can be grown on a copper foil. For this reason, for example, the two-dimensional material, such as graphene, grown on the copper foil is transferred onto the insulating substrate 10, followed by patterning by photolithography and etching to form the first semiconductor layer 11 and the second semiconductor layer 21 on the insulating substrate 10. For the carbon nanotubes, a method of directly growing carbon nanotubes from a catalyst, such as iron, formed on an oxidized silicon substrate or a method of disposing carbon nanotubes synthesized in a different place on the substrate is used. In the example shown in FIG. 9, the first semiconductor layer 11 is formed on the insulating substrate 10 to cover one end of the first source electrode 12 and one end of the first drain electrode 13, and the second semiconductor layer 21 is formed on the insulating substrate 10 to cover one end of the second source electrode 22 and one end of the second drain electrode 23.

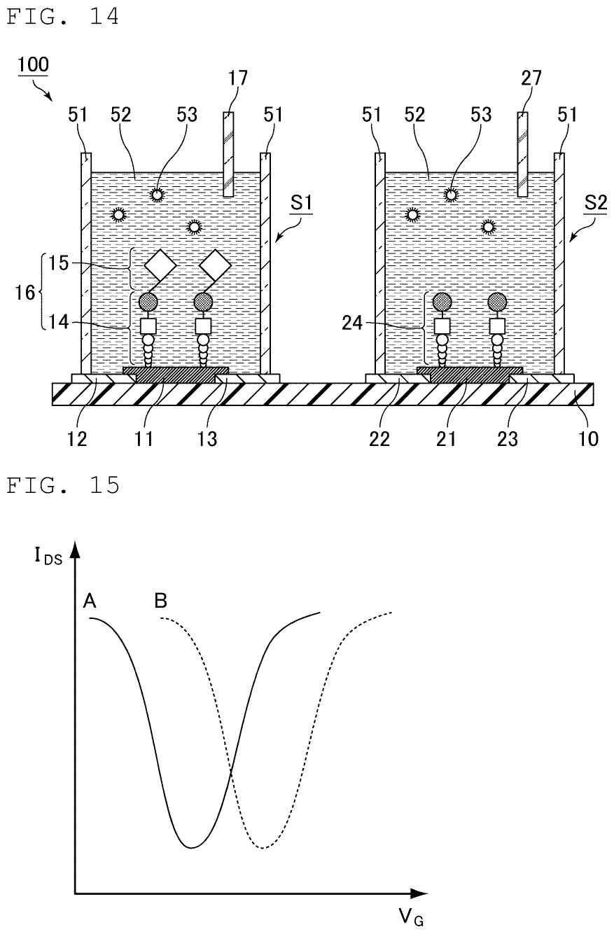

FIG. 10 is a schematic view showing one example of a step of separating between a measurement sensor region and a reference sensor region.

For example, the measurement sensor region and the reference sensor region are separated from each other by providing a partition 50 on the insulating substrate 10. As the partition 50, for example, a silicone plate called rubber pool is attached onto the insulating substrate 10.

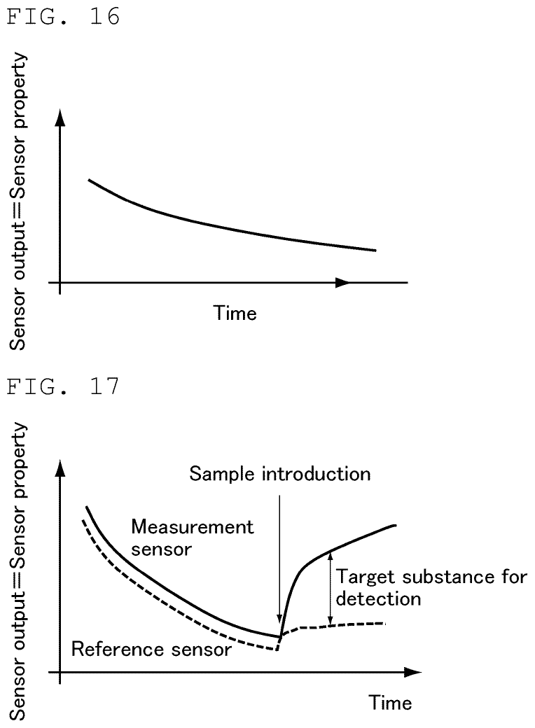

FIG. 11 is a schematic view showing one example of a step of disposing the probe molecule and the second basic moiety. For example, the measurement sensor S1 is prepared by adding a solution including the probe molecule 16 with the first basic moiety 14 and the recognition moiety 15 dropwise onto the first semiconductor layer 11 to immobilize the probe molecule 16, and the reference sensor S2 is prepared by adding a solution including the second basic moiety 24 dropwise onto the second semiconductor layer 21 to immobilize the second basic moiety 24. The solution can be added dropwise by spotting using a micropipette, an inkjet device, or a microdispenser, for example.

The biosensor 1 shown in FIG. 1 is preferably obtained through the steps above, for example. The partition 50 may be removed before the biosensor is used, or may be left during use thereof.

FIG. 12 is a schematic view showing another example of a biosensor according to a preferred embodiment of the present invention.

The biosensor 2 shown in FIG. 12 further includes a first gate electrode 17 to apply an electric field from the outside to the first semiconductor layer 11 (see FIG. 1) included in the measurement sensor S1, and a second gate electrode 27 to apply an electric field from the outside to the second semiconductor layer 21 (see FIG. 1) included in the reference sensor S2.

The first gate electrode 17 applies a potential to the first source electrode 12 and the first drain electrode 13, and a noble metal is usually used. The first gate electrode 17 is disposed in a place other than the place where the first source electrode 12 and the first drain electrode 13 are formed.

The second gate electrode 27 applies a potential to the second source electrode 22 and the second drain electrode 23, and a noble metal is usually used. The material for the second gate electrode 27 and that for the first gate electrode 17 may be the same or different. The second gate electrode 27 is disposed in a place other than the place where the second source electrode 22 and the second drain electrode 23 are formed.

In the biosensor 2 shown in FIG. 12, the current value of the measurement sensor S1 is measured with an ammeter A1, and the current value of the reference sensor S2 is measured with an ammeter A2. In other words, the current value of the measurement sensor S1 and that of the reference sensor S2 are independently measured. The ammeter A1 may be disposed closer to the first source electrode 12, or may be disposed closer to the first drain electrode 13. Similarly, the ammeter A2 may be disposed closer to the second source electrode 22, or may be disposed closer to the second drain electrode 23. In either of the configurations, the current value of the measurement sensor S1 and that of the reference sensor S2 can be measured independently.

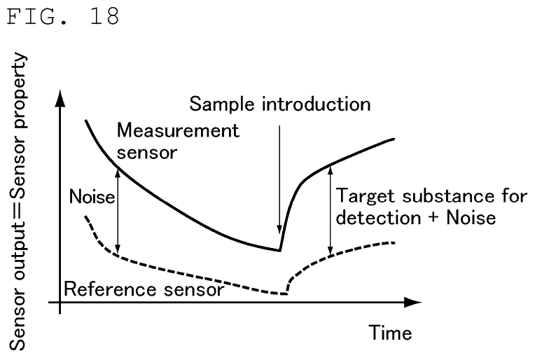

FIG. 13 is a schematic view showing further another example of a biosensor according to a preferred embodiment of the present invention.

In the biosensor 3 shown in FIG. 13, the first source electrode 12 and the second source electrode 22 are shared by the measurement sensor S1 and the reference sensor S2, the first drain electrode 13 and the second drain electrode 23 are shared by the measurement sensor S1 and the reference sensor S2, and the first gate electrode 17 and the second gate electrode 27 are shared by the measurement sensor S1 and the reference sensor S2.

In the biosensor 3 shown in FIG. 13, the current value of the measurement sensor S1 is measured with an ammeter A1, and that of the reference sensor S2 is measured with an ammeter A2. In other words, the current value of the measurement sensor S1 and that of the reference sensor S2 are independently measured. The ammeter A1 may be disposed closer to the first source electrode 12, or may be disposed closer to the first drain electrode 13. Similarly, the ammeter A2 may be disposed closer to the second source electrode 22, or may be disposed closer to the second drain electrode 23. In either of the configurations, the current value of the measurement sensor S1 and that of the reference sensor S2 can be measured independently.

In a biosensor according to a preferred embodiment of the present invention, the source electrode, the drain electrode, and the gate electrode may be separately disposed for each of the measurement sensor and the reference sensor, or may be shared (or potentially equalized). The electrodes separately disposed for each of the measurement sensor and the reference sensor and the electrodes shared thereby may be mixed. Even in the case where these electrodes are shared, the value of the current flowing between the source electrode and the drain electrode can be read independently.

FIG. 14 is a schematic view schematically showing one example of a biosensor according to a preferred embodiment of the present invention when used.

The biosensor 100 shown in FIG. 14 includes the measurement sensor S1 and the reference sensor S2 on the insulating substrate 10. The measurement sensor S1 has a configuration in which, for example, a silicone rubber pool 51 is attached to the insulating substrate 10, the inside of the pool 51 is filled with an electrolyte solution 52, the first gate electrode 17 is impregnated with the electrolyte solution 52, and a bipotentiostat (not illustrated) is connected to the first source electrode 12, the first drain electrode 13, and the first gate electrode 17. Likewise, the reference sensor S2 has a configuration in which, for example, the silicone rubber pool 51 is attached to the insulating substrate 10, the inside of the pool 51 is filled with the electrolyte solution 52, the second gate electrode 27 is impregnated with the electrolyte solution 52, and a bipotentiostat (not illustrated) is connected to the second source electrode 22, the second drain electrode 23, and the second gate electrode 27. The electrolyte solution 52 includes a target substance for detection (target molecule) 53.

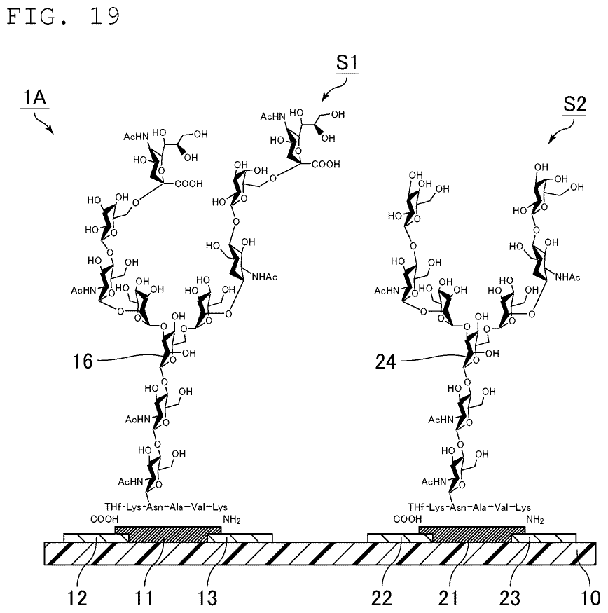

FIG. 15 is a graph showing the relation between a gate voltage V_{G} and a source-drain current I_{DS}.

In FIG. 15, a solid line A represents the source-drain current I_{DS} when the probe molecule is not bound to the target substance for detection, and a dashed line B represents the source-drain current I_{DS} when the probe molecule is bound to the target substance for detection. As shown in FIG. 15, when the probe molecule specifically binds to the target substance for detection, the conduction properties are modulated by the charge of the target molecule as the target substance for detection. By monitoring the modulation, the presence of the target substance for detection or the concentration thereof can be sensed.

Hereinafter, the method of detecting the target substance for detection using a biosensor according to a preferred embodiment of the present invention will be described. Such a detection method is encompassed within the scope of preferred embodiments of the present invention.

Initially, a sample including the target substance for detection is fed to the measurement sensor S1 and the reference sensor S2. Thereby, the sample is brought into contact with the sensors. For example, in the case where the sample including the target substance for detection is a liquid, the sample may be added dropwise to the sensors using a dropper or the like, and may be introduced into the sensors through a flow path.

Examples of the sample including the target substance for detection include biological samples of subjects such as saliva, throat swabs, nasal discharge, tear fluid, biological fluids (such as blood), urine, and feces, cell or virus suspensions, drinking water, sewage, and exhaled air. The sample including the target substance for detection need not to be a liquid.

Next, in the measurement sensor S1, the value of a first current flowing between the first source electrode 12 and the first drain electrode 13 is measured by applying a voltage between the first source electrode 12 and the first drain electrode 13.

In the reference sensor S2, the value of a second current flowing between the second source electrode 22 and the second drain electrode 23 is measured by applying a voltage between the second source electrode 22 and the second drain electrode 23.

The property of the measurement sensor S1 is then corrected by comparing the property of the measurement sensor S1 obtained from the value of the first current to the property of the reference sensor S2 obtained from the value of the second current or by comparing a temporal change in the property of the measurement sensor S1 to a temporal change in the property of the reference sensor S2.

Examples of the combination of the property of the measurement sensor S1 with the property of the reference sensor S2 include the following sensor properties in the state where the voltage applied between the first source electrode 12 and the first drain electrode 13 and the voltage applied between the second source electrode 22 and the second drain electrode 23 are kept constant:
Sensor property 1: a combination of the value of the voltage applied between the first gate electrode 17 and the first source electrode 12 at the minimum value of the first current with the value of the voltage applied between the second gate electrode 27 and the second source electrode 22 at the minimum value of the second current.
Sensor property 2: a combination of the current value which is the minimum value of the first current with the current value which is the minimum value of the second current.
Sensor property 3: a combination of the value of the first current with the value of the second current when the value of the first gate voltage applied between the first gate electrode 17 and the first source electrode 12 and the value of the second gate voltage applied between the second gate electrode 27 and the second source electrode 22 are constant.

Among these sensor properties above, the sensor properties 1 and 2 are obtained in a measurement mode in which the gate voltage between the source electrode and the gate electrode are repeatedly swept. This measurement mode is advantageous in that a large quantity of information is obtained.

Among these sensor properties above, the sensor property 3 is obtained in a measurement mode in which the gate voltage between the source electrode and the gate electrode is fixed. This measurement mode is advantageous in that temporal resolution of measurement is high.

FIG. 16 is a graph showing one example of the temporal change in sensor property.

As shown in FIG. 16, the temporal change in sensor property is obtained as the output of the sensor.

In the step of correcting the property of the measurement sensor S1, the temporal change in property of the measurement sensor S1 may be compared to the temporal change in property of the reference sensor S2, or the property of the measurement sensor S1 at one time point may be compared to the property of the reference sensor S2 at that time point.

In the comparison, these properties may be compared using the difference between the outputs of the sensors or the proportions of the outputs thereof, for example.

Subsequently, the result of comparison between the outputs of the sensors is presented.

FIG. 17 is a graph showing one example of the outputs of the measurement sensor and the reference sensor by the detection method using a biosensor according to a preferred embodiment of the present invention. FIG. 18 is a graph showing one example of the outputs of the measurement sensor and the reference sensor by the detection method using a conventional biosensor.

In the reference sensor S2 included in a biosensor according to a preferred embodiment of the present invention, the recognition moiety 15 is absent at the distal end of the second basic moiety 24, and the second basic moiety 24 has the same structure as that of the first basic moiety 14 of the probe molecule 16. For this reason, as shown in FIGS. 17 and 18, by comparing the output of the measurement sensor S1 to that of the reference sensor S2, a variation attributed to noises can be reduced, enhancing the measurement precision and reliability.

In a detection method according to a preferred embodiment of the present invention, desirably a background is measured using a sample without the target substance for detection before the sample including the target substance for detection is fed to the measurement sensor S1 and the reference sensor S2.

A method of measuring a background includes feeding a sample without the target substance for detection to the measurement sensor and the reference sensor, measuring the value of the first current flowing between the first source electrode and the first drain electrode in the measurement sensor by applying a voltage between the first source electrode and the first drain electrode, measuring the value of the second current flowing between the second source electrode and the second drain electrode in the reference sensor by applying a voltage between the second source electrode and the second drain electrode, and correcting the property of the measurement sensor by comparing the property of the measurement sensor obtained from the value of the first current to the property of the reference sensor obtained from the value of the second current or by comparing a temporal change in the property of the measurement sensor to a temporal change in the property of the reference sensor.

When the background is measured, the sample without the target substance for detection is removed from the measurement sensor S1 and the reference sensor S2 before the sample including the target substance for detection is fed to the measurement sensor S1 and the reference sensor S2.

A device for detecting a target substance for detection using a biosensor according to a preferred embodiment of the present invention is also encompassed within the scope of preferred embodiments of the present invention.

Specifically, a detection device according to a preferred embodiment of the present invention is a device for detecting a target substance for detection using a biosensor according to a preferred embodiment of the present invention, the device including a first current calculator to measure a value of the first current flowing between the first source electrode and the first drain electrode in the measurement sensor by applying a voltage between the first source electrode and the first drain electrode, a second current calculator to measure a value of the second current flowing between the second source electrode and the second drain electrode in the reference sensor by applying a voltage between the second source electrode and the second drain electrode, and a corrector to correct a property of the measurement sensor by comparing a property of the measurement sensor obtained from the value of the first current to a property of the reference sensor obtained from the value of the second current or by comparing a temporal change in the property of the measurement sensor to a temporal change in the property of the reference sensor.

Hereinafter, preferred embodiments of a biosensor according to a preferred embodiment of the present invention will be described.

The preferred embodiments shown below are exemplary, and the configurations shown in different preferred embodiments can be partially replaced or combined. In a second preferred embodiment and thereafter, descriptions of things shared with the first preferred embodiment will be omitted, and only differences will be described. In particular, similar effects of similar configurations will not be mentioned point by point in each of the preferred embodiments.

### First Preferred Embodiment

In a biosensor according to a first preferred embodiment of the present invention, a sialoglycan as the probe molecule is disposed in the measurement sensor, and an asialoglycan as the reference sensor is disposed in the second basic moiety.

FIG. 19 is a schematic view showing one example of a biosensor according to the first preferred embodiment of the present invention.

In the illustration of FIG. 19, the probe molecule 16 and the second basic moiety 24 are intentionally enlarged for description. FIG. 19 schematically shows differences between the structures of the sensors, and is not reflective of the actual state. The same is true in the subsequent drawings.

The biosensor 1A shown in FIG. 19 includes the measurement sensor S1 and the reference sensor S2 on the insulating substrate 10. In the biosensor 1A, α2,6-sialylglycopeptide as the probe molecule 16 is disposed in the measurement sensor S1, and asialoglycopeptide as the second basic moiety 24 is disposed in the reference sensor S2. In the measurement sensor S1, instead of α2,6-sialylglycopeptide, α2,3-sialylglycopeptide may be disposed as the probe molecule 16.

### Second Preferred Embodiment

In a biosensor according to a second preferred embodiment of the present invention, the probe molecule is disposed in the measurement sensor via a linker molecule which is present on the surface of the first semiconductor layer and which includes a pyrenyl group. Furthermore, the second basic moiety is preferably disposed in the reference sensor via the linker molecule which is present on the surface of the second semiconductor layer and which includes a pyrenyl group.

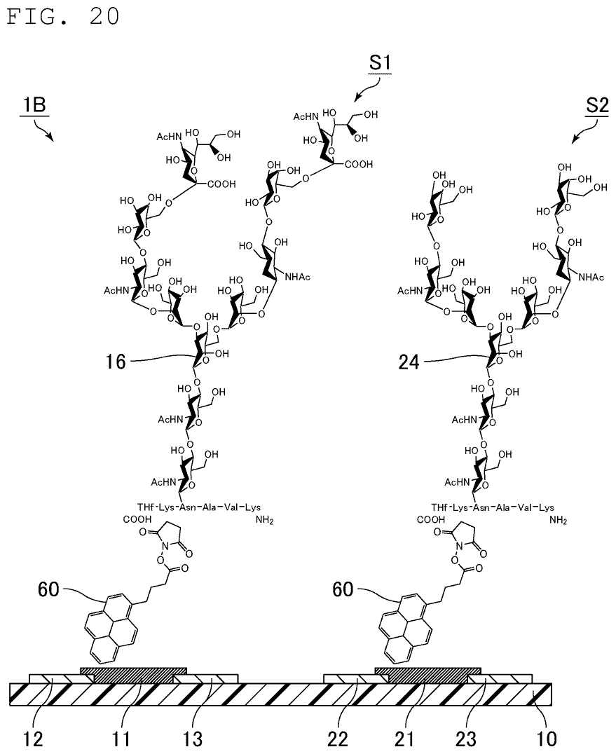

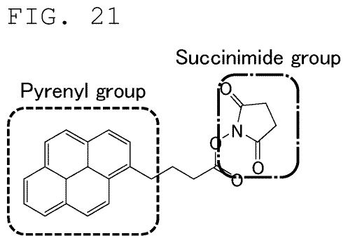

FIG. 20 is a schematic view showing one example of a biosensor according to a second preferred embodiment of the present invention.

In the biosensor 1B shown in FIG. 20, the probe molecule 16 is disposed in the measurement sensor S1 via a linker molecule which is present on the surface of the first semiconductor layer 11 and which includes a pyrenyl group 60. Furthermore, the second basic moiety 24 is disposed in the reference sensor S2 via the linker molecule which is present on the surface of the second semiconductor layer 21 and which includes a pyrenyl group 60.

FIG. 21 is a diagram showing one example of the structure of the linker molecule including a pyrenyl group.

FIG. 21 shows 1-pyrenebutanoic acid succinimidyl ester (PBASE).

As shown in FIG. 21, because a pyrenyl group including four benzene rings planarly arranged is present at one end of PBASE, PBASE can bind to the surface of the layer of a carbon-based semiconductor such as graphene via a non-covalent bond called n-n interaction. Because a succinimide group is present at the other end of PBASE, PBASE can bind to the modified amino group at the terminal of the probe molecule such as a glycan. Therefore, compared to the case where the probe molecule such as a glycan is disposed on the surface of the semiconductor layer via a covalent bond, this configuration can reduce influences on the crystal structure of the semiconductor layer, thus reducing degradation of the properties.

### Third Preferred Embodiment

A biosensor according to a third preferred embodiment of the present invention includes a plurality of measurement sensors, and different probe molecules are disposed in the respective measurement sensors. Use of a plurality of measurement sensors can improve precision in identification of the target molecule.

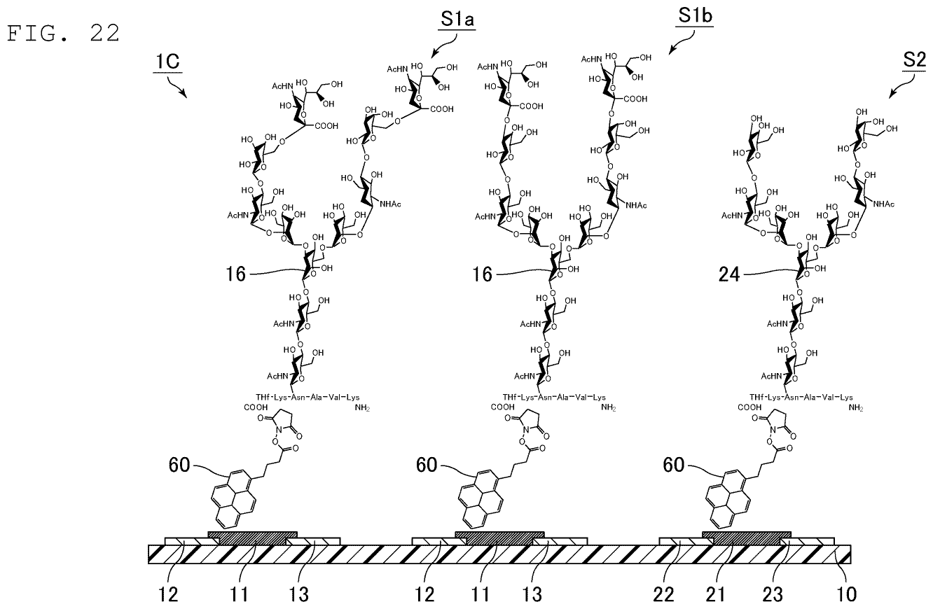

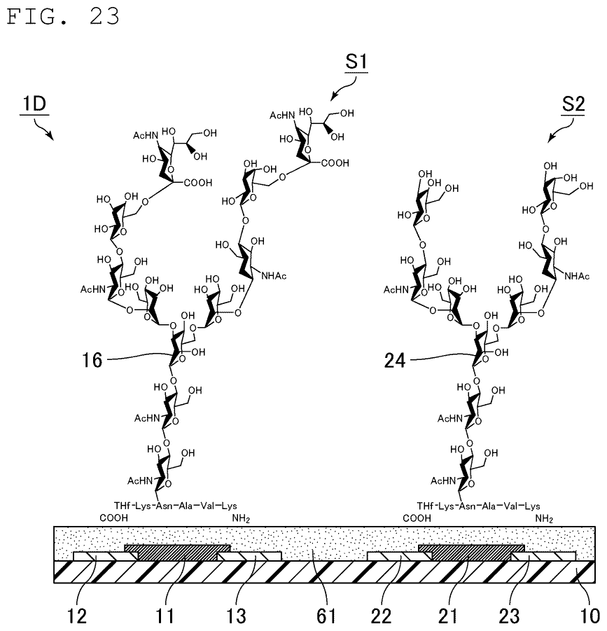

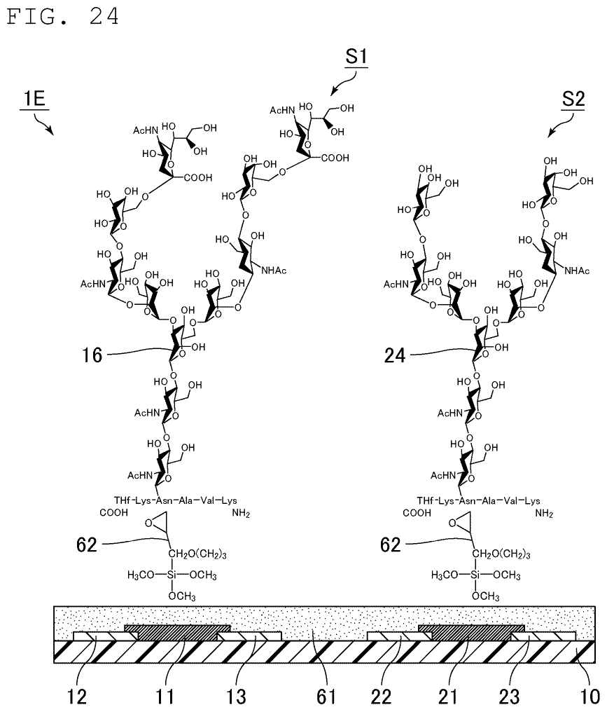

FIG. 22 is a schematic view showing one example of a biosensor according to a third preferred embodiment of the present invention.

A biosensor 1C shown in FIG. 22 includes a first measurement sensor S1a, a second measurement sensor Sib, and a reference sensor S2 on the insulating substrate 10. In the biosensor 1C, α2,6-sialylglycopeptide as the probe molecule 16 is disposed in the first measurement sensor S1a, α2,3-sialylglycopeptide as the probe molecule 16 is disposed in the second measurement sensor Sib, and asialoglycopeptide as the second basic moiety 24 is disposed in the reference sensor S2. The linker molecule 60 including a pyrenyl group need not to be present in these sensors.

### Fourth Preferred Embodiment

In a biosensor according to a fourth preferred embodiment of the present invention, the probe molecule is disposed in the measurement sensor via an oxidized film present on the surface of the first semiconductor layer. Furthermore, the second basic moiety is preferably disposed in the reference sensor via the oxidized film present on the surface of the second semiconductor layer.

The surface of the semiconductor layer is hydrophilicized by covering the surface of the semiconductor layer with the oxidized film. This reduces non-specific adsorption, increasing precision.

FIG. 23 is a schematic view showing one example of a biosensor according to a fourth preferred embodiment of the present invention.

In the biosensor 1D shown in FIG. 23, the probe molecule 16 is disposed in the measurement sensor S1 via an oxidized film 61 present on the surface of the first semiconductor layer 11. Furthermore, the second basic moiety 24 is disposed in the reference sensor S2 via the oxidized film 61 present on the surface of the second semiconductor layer 21.

In the example shown in FIG. 23, the oxidized film 61 is disposed across the insulating substrate 10 to cover the surface of the first semiconductor layer 11 and the surface of the second semiconductor layer 21. Although the oxidized film 61 may be disposed on the first source electrode 12, the first drain electrode 13, the second source electrode 22, and the second drain electrode 23 as shown in FIG. 23, it is sufficient that the oxidized film 61 is disposed on the insulating substrate 10 to cover at least the surface of the first semiconductor layer 11 and the surface of the second semiconductor layer 21.

Examples of oxides forming the oxidized film 61 include SiO₂, Al₂O₃, TiO₂, HfO₂, ZrO², SiNₓ, and composite oxides thereof.

The oxide forming the oxidized film 61 can be verified by elemental analysis of the surface of the sensor by X-ray photoemission spectroscopy (XPS). Alternatively, it can also be verified by elemental analysis of the surface of the sensor by energy dispersive X-ray spectroscopy (EDS).

Examples of a method of forming the oxidized film 61 include methods such as deposition, sputtering, atomic layer deposition (ALD), thermal chemical vapor deposition (thermal CVD), and catalyst chemical vapor deposition (catalyst CVD).

The oxidized film 61 preferably has a thickness of about 2 nm or more, for example, from the viewpoint of ensuring electrical insulation of the surface of the sensor and mechanical stability (e.g., mechanical stability to ultrasonic washing) of the oxidized film 61. On the other hand, the oxidized film 61 preferably has a thickness of about 30 nm or less, for example. An oxidized film 61 having a thickness of about 30 nm or less, for example, ensures high sensitivity of the sensor.

The thickness of the oxidized film 61 can be measured by observation of a cross-section with a transmission electron microscope (TEM).

The oxidized film 61 preferably includes amorphous regions. Such an oxidized film can enhance the electrical insulation of the surface of the sensor, compared to the case where the entire oxidized film 61 is crystalline. In the case where the oxidized film 61 includes amorphous regions, the oxidized film 61 need not to be completely amorphous, and may partially include crystalline regions.

The amorphous regions, when included in the oxidized film 61, can be verified by crystallinity analysis of an X-ray diffraction image or an electron diffraction image obtained in measurement with a transmission electron microscope (TEM).

In a biosensor according to the fourth preferred embodiment of the present invention, preferably, the probe molecule is disposed in the measurement sensor via a silane coupling agent present on the surface of the oxidized film. Furthermore, preferably, the second basic moiety is disposed in the reference sensor via the silane coupling agent present on the surface of the oxidized film.

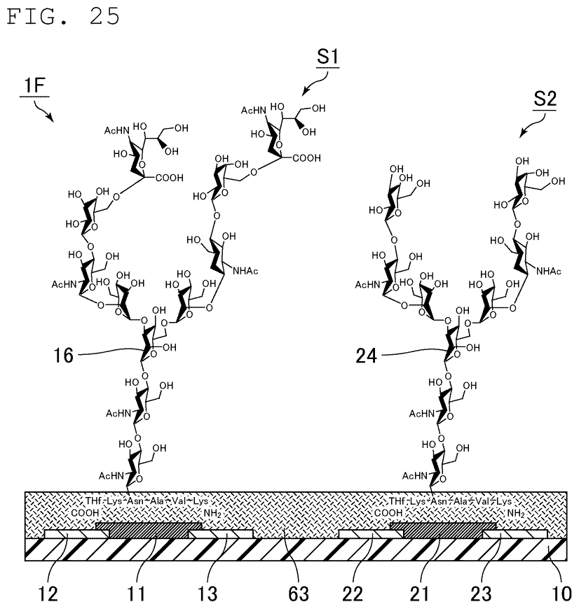

FIG. 24 is a schematic view showing another example of a biosensor according to the fourth preferred embodiment of the present invention.

In the biosensor 1E shown in FIG. 24, the probe molecule 16 is disposed in the measurement sensor S1 via a silane coupling agent 62 present on the surface of the oxidized film 61. Furthermore, the second basic moiety 24 is disposed in the reference sensor S2 via the silane coupling agent 62 present on the surface of the oxidized film 61.

When the silane coupling agent 62 is present on the surface of the oxidized film 61, the oxidized film 61 strongly binds to the silane coupling agent 62 via a covalent bond, and the silane coupling agent 62 strongly binds to the probe molecule 16 via a covalent bond. Such a configuration can make it difficult for the probe molecule 16 to come off, enhancing the reliability of the sensor.

Examples of the silane coupling agent 62 include silane coupling agents having an amino group, such as 3-aminopropyltriethoxysilane (APTES) and 3-aminopropyltrimethoxysilane (APTMS); silane coupling agents having a thiol group, such as 3-mercaptopropyltriethoxysilane (MPTES); and silane coupling agents having an epoxy group such as triethoxy(3-glycidyloxypropyl)silane (GPTES).

The presence of the silane coupling agent 62 on the surface of the oxidized film 61 can be verified by surface analysis using time-of-flight secondary ion mass spectrometry (TOF-SIMS).

The silane coupling agent 62 can be replaced by another material as long as it is a material which forms a covalent bond on the oxidized film 61. Specific examples of such a material include phosphonic acid derivatives.

In a biosensor according to the fourth preferred embodiment of the present invention, the probe molecule may be disposed in the measurement sensor via a spacer molecule present on the surface of the oxidized film. Furthermore, the second basic moiety may be disposed in the reference sensor via the spacer molecule present on the surface of the oxidized film.

When the spacer molecule is present on the surface of the oxidized film, the probe molecule disposed in the measurement sensor is spaced from the surface of the oxidized film by the spacer molecule to have freedom. This results in improved sensing ability of the probe molecule. When the spacer molecule has hydrophilicity, the hydrophilicity of the surface of the sensor can be increased.

Examples of the spacer molecule include polyethylene glycol (PEG), polyvinylpyrrolidone (PVP), dextran, and ethylene glycol bis(succinimidyl succinate). The arm length of the spacer molecule is desirably about 0.7 nm or more and about 10 nm or less, for example, although not limited thereto.

In a biosensor according to the fourth preferred embodiment of the present invention, a seed layer may be disposed between the first semiconductor layer and the oxidized film. Furthermore, the seed layer may be disposed between the second semiconductor layer and the oxidized film. The presence of the seed layer allows uniform formation of the oxidized film, enhancing the sensitivity of the biosensor.

The seed layer can be formed by forming a film of a light metal such as aluminum (Al) or magnesium (Mg), a 3d transition metal such as titanium (Ti), nickel (Ni), or chromium (Cr), or a rare metal such as hafnium (Hf), zirconium (Zr), or yttrium (Y) in the form of an elemental metal, and oxidizing the film.

The seed layer preferably has a thickness of about 2 nm or less, for example. On the other hand, the seed layer preferably has a thickness of about 0.5 nm or more, for example.

In a biosensor according to the fourth preferred embodiment of the present invention, the surface of the oxidized film may have depressions and projections. The depressions and projections disposed on the surface of the oxidized film can increase the surface area of the oxidized film, thus increasing the hydrophilicity of the surface of the sensor. These can also increase the density of the probe molecules disposed in the measurement sensor, thus enhancing the sensitivity of the biosensor.

Examples of the method of forming depressions and projections on the surface of the oxidized film include a method of roughening the surface of the oxidized film by surface blasting or plasma asking, and a method by forming the oxidized film by island growth. The island growth is a phenomenon that nuclei separated from each other grow therefrom. The film is unevenly formed by island growth, and as a result, depressions and projections are formed on the surface of the film. The requirement for island growth includes that the surface of the underlying layer of the film to be grown has poor wettability to the raw material of the film.

### Fifth Preferred Embodiment

In a biosensor according to a fifth preferred embodiment of the present invention, a blocking agent together with the probe molecule is present in the measurement sensor. Furthermore, preferably, the blocking agent together with the second basic moiety is present in the reference sensor. The blocking agent increases the hydrophilicity of the surface of the sensor. As a result, non-specific adsorption is reduced, thus increasing precision.

FIG. 25 is a schematic view showing one example of a biosensor according to the fifth preferred embodiment of the present invention.

In the biosensor 1F shown in FIG. 25, a blocking agent 63 together with the probe molecule 16 is present in the measurement sensor S1. Furthermore, the blocking agent 63 together with the second basic moiety 24 is present in the reference sensor S2.

Examples of the blocking agent 63 include proteins (such as bovine serum albumin (BSA), hemoglobin, and skim milk), surfactants (such as Tween (trade name), Triton (trade name), and sodium dodecyl sulfate (SDS)), and polymers (such as PEG and PVP). These blocking agents may be used alone or as the mixture thereof. The proteins used as the blocking agent 63 include a large number of sugar proteins having a glycan on their surfaces. For example, BSA has sialic acid on its surface. For this reason, in the case where a sugar protein is used as the blocking agent 63 in the measurement sensor S1 where the glycan is disposed as the probe molecule 16, the target molecule may actively adhere to the sugar protein of the blocking agent 63. In consideration of this, preferably, no sugar protein is used as the blocking agent 63 when a glycan is used as the probe molecule 16.

The biosensors, the detection methods, and the detection devices according to the present invention are not limited to the preferred embodiments above, and the configuration of the biosensors and the conditions for producing the biosensors can be subjected to a variety of modifications and changes within the scope of the present invention.

For example, in a biosensor according to a preferred embodiment of the present invention, an insulating coating layer may be disposed in a portion other than the sensing portion of the measurement sensor. Furthermore, the insulating coating layer may be disposed in a portion other than the sensing portion of the reference sensor. The insulating coating layer, when disposed, enhances the insulation of the portion other than the sensing portion, and thus improves the reliability of the biosensor. The insulating coating layer prevents trapping of the target molecule in the portion other than the sensing portion, thus enhancing the sensitivity of the biosensor.

Examples of the material for forming the insulating coating layer include organic compounds such as polyimide, epoxy resins, acrylic resins, and fluorinated resins. The insulating coating layer desirably has a thickness of about 100 nm or more and about 10 μm or less, for example.

Alternatively, in a biosensor according to a preferred embodiment of the present invention, the insulating coating layer may be disposed on the first source electrode and the first drain electrode, and the first semiconductor layer may be disposed on the first source electrode, the first drain electrode, and the insulating coating layer. Furthermore, the insulating coating layer may be disposed on the second source electrode and the second drain electrode, and the second semiconductor layer may be disposed on the second source electrode, the second drain electrode, and the insulating coating layer.

While preferred embodiments of the present invention have been described above, it is to be understood that variations and modifications will be apparent to those skilled in the art without departing from the scope and spirit of the present invention. The scope of the present invention, therefore, is to be determined solely by the following claims.

What is claimed is:

1. A field-effect transistor-based biosensor comprising:
an insulating substrate; and
a measurement sensor and a reference sensor on the insulating substrate; wherein
the measurement sensor includes a first semiconductor layer, a first source electrode, and a first drain electrode, the first source electrode and the first drain electrode being electrically connected to the first semiconductor layer;
the reference sensor includes a second semiconductor layer, a second source electrode, and a second drain electrode, the second source electrode and the second drain electrode being electrically connected to the second semiconductor layer;
a probe molecule is in the measurement sensor;
the probe molecule has a first basic moiety in the measurement sensor, and a recognition moiety with a first end bound to the first basic moiety and a second end defining a distal end of the probe molecule;
a second basic moiety is in the reference sensor, the second basic moiety having a same structure as that of the first basic moiety of the probe molecule in the measurement sensor; and
the recognition moiety of the probe molecule in the measurement sensor is absent at the distal end of the second basic moiety.

2. The biosensor according to claim 1, wherein the first basic moiety and the second basic moiety both are glycans.

3. The biosensor according to claim 1, wherein
the first basic moiety and the second basic moiety both are an asialoglycan; and
the probe molecule is a sialoglycan.

4. The biosensor according to claim 3, wherein the sialoglycan is an α2,3-linked sialic acid-including glycan and/or an α2,6-linked sialic acid-including glycan.

5. The biosensor according to claim 4, wherein
the α2,3-linked sialic acid-including glycan is an α2,3-linked sialic acid-including glycan binding peptide;
the α2,6-linked sialic acid-including glycan is an α2,6-linked sialic acid-including glycan binding peptide; and
the asialoglycan is an asialoglycan-binding peptide.

6. The biosensor according to claim 1, wherein the first semiconductor layer includes graphene or carbon nanotubes.

7. The biosensor according to claim 6, wherein the probe molecule is provided in the measurement sensor via a linker molecule located on a surface of the first semiconductor layer and including a pyrenyl group.

8. The biosensor according to claim 6, wherein the probe molecule is provided in the measurement sensor via an oxidized film on a surface of the first semiconductor layer.

9. The biosensor according to claim 8, wherein the probe molecule is provided in the measurement sensor via a silane coupling agent on a surface of the oxidized film.

10. The biosensor according to claim 6, wherein a blocking agent together with the probe molecule is present in the measurement sensor.

11. The biosensor according to claim 1, further comprising:
a first gate electrode to apply an electric field from an outside to the first semiconductor layer; and
a second gate electrode to apply an electric field from an outside to the second semiconductor layer.

12. The biosensor according to claim 1, wherein the biosensor is structured and operable to detect influenza virus, reovirus, adenovirus, rotavirus, or novel coronavirus.

13. A method of detecting a target substance for detection using the biosensor according to claim 1, the method comprising:
feeding a sample including a target substance for detection to the measurement sensor and the reference sensor;
measuring a value of a first current flowing between the first source electrode and the first drain electrode in the measurement sensor by applying a voltage between the first source electrode and the first drain electrode;
measuring a value of a second current flowing between the second source electrode and the second drain electrode in the reference sensor by applying a voltage between the second source electrode and the second drain electrode; and
correcting a property of the measurement sensor by comparing a property of the measurement sensor obtained from the value of the first current to a property of the reference sensor obtained from the value of the second current or by comparing a temporal change in the property of the measurement sensor to a temporal change in the property of the reference sensor.

14. The method according to claim 13, wherein
the biosensor further includes a first gate electrode to apply an electric field from an outside to the first semiconductor layer, and a second gate electrode to apply an electric field from an outside to the second semiconductor layer; and
a combination of the property of the measurement sensor with the property of the reference sensor includes the following sensor properties in a state where a voltage applied between the first source electrode and the first drain electrode and a voltage applied between the second source electrode and the second drain electrode are kept constant:
a combination of a value of the voltage applied between the first gate electrode and the first source electrode at a minimum value of the first current with a value of the voltage applied between the second gate electrode and the second source electrode at a minimum value of the second current;
a combination of the current value which is the minimum value of the first current with the current value which is the minimum value of the second current; or
a combination of the value of the first current with the value of the second current when the value of a first gate voltage applied between the first gate electrode and the first source electrode and the value of a second gate voltage applied between the second gate electrode and the second source electrode are constant.

15. The method according to claim 13, wherein the method detects influenza virus, reovirus, adenovirus, rotavirus, or novel coronavirus.

16. The method according to claim 13, wherein the sample is one of saliva, throat swabs, nasal discharge, tear fluid, a biological fluid, urine, feces, a cell suspension, a virus suspension, drinking water, sewage, or exhaled air.

17. A device for detecting a target substance, the device comprising:
the biosensor according to claim 1;
a first current calculator to measure a value of the first current flowing between the first source electrode and the first drain electrode in the measurement sensor by applying a voltage between the first source electrode and the first drain electrode;
a second current calculator to measure a value of the second current flowing between the second source electrode and the second drain electrode in the reference sensor by applying a voltage between the second source electrode and the second drain electrode; and
a corrector to correct a property of the measurement sensor by comparing a property of the measurement sensor obtained from the value of the first current to a property of the reference sensor obtained from the value of the second current or by comparing a temporal change in the property of the measurement sensor to a temporal change in the property of the reference sensor.

18. The device according to claim 17, wherein the device is structured and operable to detect influenza virus, reovirus, adenovirus, rotavirus, or novel coronavirus.
